SEQUENCE LISTING (5 sequences)

SEQ ID NO: 1 - DNA, 11569 bp, Artificial Sequence
  Adenoviral expression plasmid pAd.3r.hscIL-12 for regulated expression of human IL-12
ctgctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctaggatcgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtggacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagagggtctgggtctctttggcatggtcgaattaattcgcgtcgagccggccgcgggtacaattccggttggacctgggagtggacacctgtggagagaaaggcaaagtggatgtcattgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagtcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacaggcgtcgaagcttgatgggtcgctcggtgttcgaggccacacgcgtcaccttaatatgcgaagtggacctcggaccgcgccgccccgactgcatctgcgtgttcgaattgcccggcgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacggtcgagggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattgcgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtaccgagctcggatccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgcctctcgagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtcgacatcgatgctagagtggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcttatcatgacattaacctataaaaataggcgtatcacgaggccctttcgtcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctctag

SEQ ID NO: 2 - DNA, 11458 bp, Artificial Sequence
  Adenoviral expression plasmid pAd.3r.mscIL-12 for regulated expression of murine IL-12
ctgctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctaggatcgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtggacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagagggtctgggtctctttggcatggtcgaattaattcgcgtcgagccggccgcgggtacaattccggttggacctgggagtggacacctgtggagagaaaggcaaagtggatgtcattgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagtcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacaggcgtcgaagcttgatgggtcgctcggtgttcgaggccacacgcgtcaccttaatatgcgaagtggacctcggaccgcgccgccccgactgcatctgcgtgttcgaattgcccggcgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacggtcgagggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattgcgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgaatggccatgggtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagaattgatccggattagtccaatttgttaaagacaggatgggccctatatatggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtcgacatcgatgctagagtggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcttatcatgacattaacctataaaaataggcgtatcacgaggccctttcgtcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctctag

SEQ ID NO: 3 - DNA, 11453 bp, Artificial Sequence
  Adenoviral expression plasmid pShuttle.3r.hscIL-12 for regulated expression of human IL-12 following virus generation using AdEasy
ctgctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctaggatcgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtggacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagagggtctgggtctctttggcatggtcgaattaattcgcgtcgagccggccgcgggtacaattccggttggacctgggagtggacacctgtggagagaaaggcaaagtggatgtcattgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagtcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacaggcgtcgaagcttgatgggtcgctcggtgttcgaggccacacgcgtcaccttaatatgcgaagtggacctcggaccgcgccgccccgactgcatctgcgtgttcgaattgcccggcgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacggtcgagggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattgcgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtaccgagctcggatccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgcctctcgagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaagtttttbtaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactg

SEQ ID NO: 4 - DNA, 9784 bp, Artificial
  Adenoviral expression plasmid pAd.3r for regulated expression
ctgctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctaggatcgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtggacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagagggtctgggtctctttggcatggtcgaattaattcgcgtcgagccggccgcgggtacaattccggttggacctgggagtggacacctgtggagagaaaggcaaagtggatgtcattgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagtcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacaggcgtcgaagcttgatgggtcgctcggtgttcgaggccacacgcgtcaccttaatatgcgaagtggacctcggaccgcgccgccccgactgcatctgcgtgttcgaattgcccggcgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacggtcgagggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattgcgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtcgacatcgatgctagagtggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcttatcatgacattaacctataaaaataggcgtatcacgaggccctttcgtcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctctag

SEQ ID NO: 5 - DNA, 9668 bp, Artificial
  Expression plasmid pShuttle.3r for regulated expression following virus generation using AdEasy
ctgctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctaggatcgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgatagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataacgcgttctctagtgaaaaaccttgttggcataaaaaggctaattgattttcgagagtttcatactgtttttctgtaggccgtgtatctgaatgtacttttgctccattgcgatgacttagtaaagcacatctaaaacttttagcgttattgcgtaaaaaatcttgccagctttccccttttaaagggcaaaagtgagtatggtgcctatctaacatctcaatggctaaggcgtcgagcaaagcccgcttattttttacatgccaatacagtgtaggctgctctacaccaagcttctgggcgagtttacgggttgttaaaccttcgattccgacctcattaagcagctctaatgcgctgttaatcactttacttttatctaatctagagggtctgggtctctttggcatggtcgaattaattcgcgtcgagccggccgcgggtacaattccggttggacctgggagtggacacctgtggagagaaaggcaaagtggatgtcattgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagtcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacaggcgtcgaagcttgatgggtcgctcggtgttcgaggccacacgcgtcaccttaatatgcgaagtggacctcggaccgcgccgccccgactgcatctgcgtgttcgaattgcccggcgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacggtcgagggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattgcgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaagtttttbtaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactg

(19) United States
(12) Patent Application Publication
(10) Pub. No.: US 2005/0233457 A1
(43) Pub. Date: Oct. 20, 2005

(54) Recombining viral vectors for the tetracycline-regulated expression of genes

(76) Inventor: Andreas Block, Hamburg (DE)

Correspondence Address: MCDERMOTT, WILL & EMERY, 4370 LA JOLLA VILLAGE DRIVE, SUITE 700, SAN DIEGO, CA, 92122, US

(21) Appl. No.: 10/511,237

(22) PCT Filed: Apr. 8, 2003
(86) PCT No.: PCT/EP03/03638

§ 371 Date: Apr. 28, 2005

(30) Foreign Application Priority Data
Apr. 15, 2002 (DE) 10216800.8
May 17, 2002 (DE) 10222513.3

(51) Int. Cl. C12N 15/86

(52) U.S. Cl. 435/456

(57) ABSTRACT

The invention relates to recombining viral vectors which can be suppressed in a highly efficient manner by means of tetracycline or tetracycline derivatives, and the use thereof for expressing genes in eukaryotic cells, particularly within the framework of gene therapy.

The invention concerns recombinant viral vectors that can be suppressed in a highly efficient manner by tetracycline or tetracycline derivatives such as e.g. doxycycline, and their use for the realization of gene expression in eukaryotic cells, in particular within the framework of gene therapy.

Malignant diseases are one of the most frequent causes of death in man. In the case of advanced and metastasized solid tumor diseases, the therapeutic options remain very limited, with a five year survival rate of many of cancers less than 10%. Therefore, metastasized cancer represents one of the major challenges in experimental medicine. By introducing therapeutic genes into tumor cells, gene therapy approaches have opened up new perspectives in the treatment of these diseases.

Adenoviruses allow efficient transfer and expression of therapeutic genes in various tissues and cell lines. Especially the further development of recombinant adenoviral vectors has enabled experimental approaches in the adenoviral gene therapy of malignant diseases (K. Kozarsky, Curr Opin Genet Dev 3 (1993) 499-503).

Due to high efficiency of gene transfer, gene therapy approaches are currently frequently limited by toxicity as a result of uncontrolled transgene expression. Especially in adenovirally mediated expression of cytokines such as interleukin-2, interleukin-12, interleukin-18 or the tumor necrosis factor α, unexpected substantial systemic side effects may even occur in the case of intratumoral administration of these recombinant adenoviruses. So far it has not been possible to efficiently control gene expression in a manner comparable to constitutive promoters (cytomegalovirus promoter) after adenoviral infection.

Currently the Tet System developed by M. Gossen et al. represents one of the most suitable tools for controlling gene expression (M. Gossen et al., PNAS USA 89 (1992) 5547-5551; M. Gossen et al. Science 268 (1995) 1766-1769). The Tet System is based on two elements of the E. coli Tet operon. The tetracycline inducible repressor protein (tetR) is fused with the transcriptional activity domain of the Herpes simplex virus VP16. This tTA fusion protein interacts with the heptamerized tetO operator sequence resulting in the transcriptional activation of the flanking minimal promoters. The binding of tetracycline and its derivatives to the TetR domain of tTA inhibits the interaction of the fusion protein with its operator sequences leading to the down regulation of transgene expression.

The use of the original tet-regulated gene expression system in recombinant adenoviral vectors (T C Harding et al. J. Neurochem. 69 (1997) 2620-2623; T C Harding et al. Nat. Biotechnol. 16 (1998) 553-555) was limited by two major obstacles. The limited packaging capacity resulted in insufficient activation of the CMV-minimal promoter by squelching (S. Rubinchic et al. Gene Therapy 8 (2000) 875-885), and the constitutional transactivator expression led to a toxicity related to VP16.

Consequently, the aim of the present invention is to provide a gene expression system which is suitable for the gene therapy of tumor diseases without exhibiting the disadvantages known from the prior art. In particular, (adeno-) viral vectors with high transgene expression are to be provided that also provide the option of efficient down regulation of this gene expression in case of serious side effects resulting from transgene expression. Moreover, the vectors should exhibit a high degree of safety in application, especially with avoidance of VP16 related toxicity known in the art.

According to the invention, the task is achieved by means of a recombinant, viral, in particular adenoviral vector which contains an insert exhibiting the general structure
tTA-intron¹-TK⁺-TetO₇-CMV⁺-intron²-transgene
in which
TetO₇ is the heptamerized tetracycline operator
TK⁺ is the minimal thymidine kinase promoter
tTA is a nucleic acid sequence which encodes a fusion protein from the repressor protein inducible by tetracycline and the transcriptional activation domain of the Herpes simplex virus VP16,
CMV⁺ is the minimal cytomegalovirus promoter and
Transgene is a nucleic acid sequence which codes for a non-viral protein
Intron¹ is any desired non-encoding nucleic acid sequence with a length of O to approximately 1000 bp and
Intron² is any desired non-encoding nucleic acid sequence with a length of O to approximately 1000 bp

For the construction of the recombinant adenoviruses according to the invention, the tetracycline inducible repressor protein (tetR) was fused with the transcriptional activation domain of the Herpes simplex virus VP16. One of the most important aspects in this respect is no longer the inhibition by binding of tetR to the operon but the positioning of the VP16 transactivator. Accordingly, a heptamerized TetO operon with two flanking minimal promoters was used for the present invention. This system (FIG. 1) leads to autoregulated transactivator expression through a positive feedback mechanism by one of the minimal promoters. At the same time, a therapeutic transgene is expressed by the other flanking minimal promoter. Doxycycline and tetracycline bind to the tetR component, and a change in the steric conformation leads to a loss of binding of tetR to the operator. This dissociation of the transactivator from the minimal promoters then leads to a reduction in the gene expression.

With vectors according to this invention a replication-deficient adenoviral system was designed and characterized for the first time on the basis of a vector, in which autoregulated transactivator expression takes place. This system allows a very tight control of transgene expression by addition of doxycycline in non-toxic concentrations. The high suppression of gene expression was achieved within a wide m.o.i. (multiplicity of infection) range and in different carcinoma cell lines.

The proportion of suppression depends on the concentration of the antibiotic used. Since a maximum suppression of the transgene expression was achieved with doxycycline concentrations of only 2 μg/ml, the vectors according to the invention are highly suitable for clinical applications.

According to a further embodiment, the invention relates to an above-mentioned vector in which the insert is inserted into the viral vector genome in reverse orientation, i.e. in the form of
5′-transgen-intron²-CMV⁺-TetO₇-TK⁺-intron¹-tTA-3′

Similarly, it is possible for the positions of tTA and the transgene in the insert to be inverted exclusively or additionally.

As long as the sequence elements “intron¹” and/or “intron²” are present (i.e. >0 bp), their length can vary independently from each other within the range of up to approximately 1000 bp and amount to e.g. up to 750, up to approximately 500 or up to approximately 250 bp in each case. In this case, the promoters are usually positioned within the intron sequence concerned.

According to a further alternative, the insert may additionally contain a lac repressor (lacR) between “CMV⁺” and “intron²” or between “intron²” and “transgene”, resulting in an additional option for regulation.

The transgene used is a nucleic acid encoding a fluorescence protein, luciferase, interleukin-12 (IL-12), interleukin-18 (IL-18), interleukin-2 (IL-2), tumor necrosis factor α (TNF-α) or interferon-γ (IFN-γ), preferably single-chain interleukin-12. Moreover, the invention relates to vectors in which one of the flanking promoters is used for the expression of a gene for apoptosis induction, for the expression of the BAX gene, for the expression of the FAS-L gene, a suicide gene such as thymidine kinase gene or cytosine deaminase gene or β-galactosidase gene.

Regarding the virus backbone used, an adenovirus, an adeno-associated adenovirus (AAV), a retrovirus, in particular a human immunodeficiency virus (HIV), a Herpes simplex virus, a Hepatitis B virus or a Hepatitis C virus is particularly suitable, wherein adenoviruses are particularly preferred.

In the vector according to the invention, the insert is cloned into the E1 region of a recombinant adenovirus; alternatively, the E3 and/or E4 region is also suitable.

According to a particular embodiment, the invention relates to a vector which is, for example, obtainable by homologous recombination of a viral, in particular an adenoviral plasmid and an expression plasmid with the nucleic acid sequence represented in SEQ ID No: 1, SEQ ID NO:2 or SEQ ID NO:3. In this context “SEQ ID NO:” represents the characteristic figure <400> used according to the WIPO standard ST.25.

The subject matter of the invention consists, moreover, of an expression plasmid with the nucleic acid sequence represented in SEQ ID NO:4 or SEQ ID NO:5 and its use for the production of an above-mentioned vector according to the invention.

As mentioned above, these vectors are suitable for the in vitro gene expression in eukaryotic cell lines or for use in gene therapy if the “transgene” encodes a therapeutically effective protein. It is, for example, possible for the “transgene” to be IL-12 or IL-18, with the vector being suitable for the gene therapy of malignant diseases. The malignant diseases are in particular a solid tumor.

In the case of the above mentioned applications, gene expression is regulated with tetracycline or tetracycline derivatives, in particular with doxycycline, oxytetracycline, chlorotetracycline, demeclocycline, methacycline or minocycline. Insofar as doxycycline is mentioned here, a person skilled in the art will recognize the transferability of the principle according to the invention to the above-mentioned tetracycline derivatives.

Further Advantages of the Invention:

In comparison with expression of the IL-12 heterodimer, driven by the constitutive human cytomegalovirus (HMCV) immediate-early promoter, which is widely used in the art, an up to 4000 fold increased cytokine secretion is observed in the case of the constructs developed within the framework of the invention in a large number of cancer cell lines. This unexpected effect is attributed to the interaction of the choice of promoter and the use of genetically produced and highly secretory, single chain IL-12. Surprisingly enough, the IL-12 expression in the absence of doxycycline was also superior to previously published adenovirally infected murine tumor cells using the CMV promoter for the regulation of the expression of the heterodimer or single chain mIL-12. Since the transduction of human tumor cells, in comparison with values reported earlier from preclinical or clinical experiments in the case of the constructs according to this invention, is also significantly higher, a further extremely advantageous effect is thus achieved in the present case. As a result of the possibility of reducing the adenoviral dose of the vectors according to the invention, it is possible to reduce vector-specific side effects, leading to greater safety in the clinical application.

With regard to interleukin-12 expression used according to a particular embodiment (compare below), these adenoviral vectors according to the invention further have the advantage that they satisfy all the preconditions for a successful cancer gene therapy. Thus, the formation of inhibitory p40-homodimers is reduced by expression of the single chain interleukin 12, which, compared to the heterodimeric form generally used, exhibits similar bioactivity, and is safeguarded by rapid regulation of gene expression in the 3r system of the invention by efficient secretion of single-chain interleukin-12. The extremely efficient, doxycycline-mediated suppression of the expression of bioactive, single chain interleukin 12 therefore contributes to the safety cancer gene therapy.

The system according to the invention is, moreover, characterized in that a western standard diet does not affect the sensitive tet-OFF system such that possible contamination of the food with traces of tetracycline or its derivatives presents no problem in the clinical environment.

By using the vectors according to the invention, the presence of transactivators is not required before infection with the vectors as a result of which the toxicity due to the constitutive expression of the transactivator and a reciprocal influence on or interference with the transcription by doxycycline-dependent, auto regulating gene expression is avoided. Consequently, the adenoviral vectors of the present invention represent a much more versatile and non-complicated tool in comparison with models of constitutive transactivator expression known in the state of the art.

Moreover, it is advantageous that the doxycycline regulated gene expression can take place following adenoviral infection of a large number of native mammalian cell lines or tissues. The auto regulation, moreover, causes a restriction in the unwanted transgene expression by reduced transactivator expression in the case of doxycycline mediated suppression. In comparison with approaches described in the art, the vectors according to the invention provide the advantage that, in the absence of doxycycline, a very high transgene expression can be obtained whereas the suppression of the transgene expression is not negatively affected by the addition of this antibiotic and up to 6000 fold suppression levels can be achieved.

The constructs according to the invention can consequently be used advantageously for expressing therapeutic transgenes of up to 4.8 kB, including apoptosis-inducing genes, and they consequently represent an important means for the molecular therapy of malignant diseases.

Within the framework of the present invention, it has, moreover, been surprisingly enough observed that the vectors according to the invention possess an at least 40 times higher sensitivity towards tetracycline compared with the detection limit in the standard HPLC processes. The system according to the invention is therefore also suitable for use as a sensitive tool for detecting very low tetracycline concentrations in biological, food chemical or similar samples and is consequently suitable for use in human and medico-veterinary diagnostics, for example (compare N. Schultze et al. Nat. Biotechnol. 14 (1996) 499-503). In this case, the transgene encodes a reporter protein such as e.g. luciferase or alike. The subject matter of the invention consequently also consists of the use of the vectors according to the invention, in which “a transgene” encodes a reporter protein, for detecting tetracycline or a derivative thereof such as e.g. doxycycline, in biological, food chemical or similar samples.

The invention will be explained in further detail in the following by way of examples.

### EXAMPLES

Cell Lines

HeLa and 293 human embryonal kidney cells were cultivated in HGDMEM (Gibco, Rockville, Md.). Human RT-4 bladder carcinoma cells and human colon adenocarcinoma cells HT29 were kept in McCoy medium (Gibco). MCF-7 and BT-20 human breast carcinoma cells and human colon (Colo 205 and SkCO-1) and pancreatic adenocarcinoma (Aspc-1) cell lines were grown in RPMI medium (Gibco). HepG2 human hepatocellular carcinoma cells were kept in MEM medium (Gibco). Cells were cultivated and divided according to standard procedures. All media were supplemented with 10% fetal bovine serum (FBS), 1% penicillin/streptomycin (Gibco) and 1% glutamine (Gibco). The human myeloma cell line U266 was grown in RPMI medium which had been supplemented with 15% FBS (Clontech) and 1% penicillin/streptomycin (Gibco).

### Example 1

Plasmid Construction

DNA fragments were separated by agarose gel electrophoresis and eluted from the agarose with the gel extraction kit (Qiagen, Valencia, Calif.). DH5 alpha cells were used for plasmid amplification. Plasmid DNA was prepared using a modified protocol for alkaline lysis, followed by a purification through a commercial ion exchange column according to the manufacturer's instructions (Qiagen). Before the transfection, LPS contaminations in the plasmid DNA preparations were reduced by a Triton X-117 extraction method (M. Coton et al., Gene therapy 1 (1994) 239-246). The plasmid pBIG 3r which contains the autoregulated tTA expression system has been described before (C. A. Strathdee, Gene 229 (1999) 21-29). The luciferase cDNA was obtained from the plasmid pGL3 basic (Promega, Madison, Wis.) by BglII and XbaI digestion and inserted into pBIG 3r which had been split with SpeI and BamHI resulting in pBIG 3r luc. The adenoviral plasmid pAd.CMV expression cassette was removed by digestion with XbaI and SalI after filling up with T4 DNA polymerase. PBIG 3r luc was digested with PvuII and SalI and the fragments containing the bicistronic expression cassette were ligated into the backbone of pAd.CMV.pA. The resulting adenoviral plasmid pAd3r-luc contained the bi-directional expression cassette which is flanked at its 5′ end by the 1-456 bp of the AD5 genome, including linker ITR and packaging signals and at its 3′ end by 3346-5865 bp of the AD5 genome. The expression of tTA driven by the minimal TK promoter was antiparallel and the expression of the luciferase gene driven by the minimal CMV promoter was parallel to the adenoviral E1 transcription. The luciferase gene was released from pGL3-basic by digestion with KpnI/SalI and ligated into the adenoviral expression plasmid pAd.CMV.pA resulting in pAd.CMV-luc. The cDNA of single-chain murine interleukin-12 was obtained from pSFG.IL-12.p40.L.p35 (G. J. Lieschke et al. Nat. Biotechnol. 15 (1997) 35-40) following digestion with NcoI and EcoRV. This fragment was subcloned into the NheI/SalI site of pAd.3r-luc and replaced the luciferase gene. The plasmid pAd.CMV.p40.IRES.p35 used subsequently contains the two murine IL-12 subunits which are separated by an internal ribosome entry site (IRES) of the encephalomyocarditis virus. The expression of this construct is under the control of the human cytomegalovirus (CMV) promoter element of −601 to −14 relative to the initiation of transcription.

### Example 2

Production and Amplification of Recombinant Adenoviral Vectors

Recombinant E1 deleted and E3 deleted adenoviruses were obtained and plaque purified following calcium phosphate mediated cotransfection of pAd.3r-luc, pAd.CMV-luc, pAd.3r-scIL-12 or pAd.CMV.p40.IRES.p35 with pBHG10 (A J Bett et al., PNAS USA 91 (1994) 8802-8806). The E1 deleted and E3 deleted adenoviruses were replicated in 293-cells and purified by CsCl centrifugation as previously described (F L Graham, Virology 54 (1973) 536-539). The titration of the purified viruses was carried out by plaque assay. The resulting titers for Ad.3r-luc, Ad.CMV-luc, Ad.3r-scIL12 and Ad.CMV-p40.IRES.p35 were 1.0×10¹⁰ p.f.u./ml (plaque forming units per ml), 7.5×10⁹ p.f.u./ml, 6.7×10⁹ p.f.u./ml and 8.0×10⁹ p.f.u./ml. Viral DNA was obtained (Qiagen DNA Blood Kit) for sequence analysis in order to confirm the insertion, the transactivator sequence and the orientation.

### Example 3

In Vitro Adenoviral Transfection

HT26, Colo205, SkCO-1, AsPc-1, HepG2, MCF-7, BT-20, HeLa, RT4 and U266 cells were seeded in six and twelve (U266) vial-plates at a concentration of 1×10⁶ cells per cavity for 6 hours before transfection. The larger HeLa, RT-4 and 293 cells were seeded in a concentration of 5×10⁵ cells per vial. U266 myeloma cells were allowed to grow in suspension culture and infected. Purified viral particles were diluted in media without supplementation and the cells were exposed to 500 μl of the suitable virus dilution per vial for 1 hour. After removal of the infectious supernatant, complete media which had been supplemented with different concentrations of doxycycline, were added. The media were changed every 24 hours.

### Example 4

Quantification of Transgene Expression

24 hours after infection with Ad.CMV-luc or Ad.3r-luc, the cells were harvested with 150 μl of cell culture lysis reagent according to the manufacturer's (Promega) instructions. The luciferase activity in 20 μl of cell lysate was measured using a Bertold LB9507 luminometer and luciferase assay substrate (Promega). The standard curves were produced using recombinant firefly luciferase (Promega) with had been diluted with CCLR to a concentration of 1 pg/ml to 300 ng/ml. Since rlu exhibit a saturation profile at higher concentrations, a 2-phase exponential association curve fitting was carried out using the Prism software package (GraphPad Software, Inc, San Diego, Calif.). The protein concentration was determined using the DC protein assay kit (BioRad, Hercules, Calif.).

The quantification of the single chain and heterodimeric mIL12 in cell-free supernatant following adenoviral infection of tumor cells was carried out by an IL12 p70 ELISA (OptEIA™, Pharmingen), the same immune reactivity and molecular weight being assumed for both forms. Splenocytes were isolated by means of standard methods. Splenocytes were then cultivated for three days with RPMI 1649 which was cultivated with 10% FBS, 1% penicillin/streptomycin and 1% glutamine in anti-mouse CD3-coated flasks in the presence of anti-human CD28 (5 μg/ml) in order accumulate T cells and to stimulate the mIL-2 secretion. The bioactivity was determined following the addition of a 50 fold diluted conditioned supernatant from Ad.3r-scIL12 (+/−doxycycline), Ad.CMV-p40.IRES.p35 and mock infected HT29 cells to 4×10⁴ murine splenocytes in a final volume of 125 μl for 24 hours. Murine IFN-γ was quantified in splenocyte-free supernatant using an IFN-γ ELISA (OptEIA™, Pharmingen). In order to determine the specific bioactivity, semi-logarithmic dilutions of conditioned supernatant liquor of both forms of the adenovirally expressed mIL-12 and baculovirus-expressed purified mIL12 (R&D systems) were tested for their mIL-12 immune reactivity (p70 ELISA) and IFN-γ induction in splenocytes, as described. The bioactivity of adenovirally expressed heterodimeric IL-12 can be reduced by forming inhibitory p40 homodimers, as described elsewhere. In the present case, no capture bioassay was used in order to reflect a potentially lower bioactivity in vivo.

### Example 5

Alternative Cloning Strategy

Cloning of Adenoviral Expression Plasmids for the Virus Synthesis by Means of the AdEasy System

As an alternative to the virus synthesis described, adenoviral expression plasmids were developed which permit virus generation by means of the AdEasy® system (Stratagene). For this purpose, the pShuttle vector (Stratagene) is digested with KpnI, blunted and subsequently digested with SalI. The 3r insert was isolated from pBIG3r by digestion with PvuII and SalI and ligated into the pShuttle. The resulting plasmid pShuttle3r permits the simple generation of different adenoviral vectors for doxycycline-suppressible gene expression. The human single chain interleukin-12 can subsequently be cloned by means of XhoI into the multiple cloning site of the pShuttle3r and results in pShuttle3r-hscIL12 (compare illustration).

Virus generation then takes place by homologous recombination with pAdEasy-1® in BJS 183 E. coli cells and selection for kanamycin. Following transfection of 293 cells with the recombination product, replicative recombinant adenoviral vectors are formed in this system (T He, S Zhou et al. Proc Natl Acad Sci USA 95 (5): 2509-14).

Virus production then takes place as described above in 293 cells.

### Example 6

Sodium Dodecyl Sulphate Polyacrylamide Gel Electrophoresis (SDS-Page) and Immunoblotting

After infection of HT29 colon carcinoma cells, lysates were loaded onto 15% acrylamide SDS gels following boiling in Laemmli sample buffers under reduced conditions. After electrophoretic separation, the proteins were transferred to 0.45 μm Immobilon-P (Millipore, Bedford, Mass.) and blocked with TBS-containing, 5% non-fat milk powder for 1 hour. Actin and the fusion protein tTA were detected using a rabbit anti-actin affinity isolated antigen-specific antibody (#A2066, Sigma, St. Louis, Miss.) and a mouse anti-TetR monoclonal antibody (M. Gossen et al., PNAS USA 89 (1992) 5547-5551) (#8632-1, Clontech). Following incubation for 1 hour, the blots were washed with TBS-containing 0.1% Tween-20, pH 7.5 and incubated with anti-rabbit and anti-mouse peroxidase-linked secondary antibodies (Dianova, Hamburg, Germany) for 1 hour at room temperature. Proteins were subsequently visualized after washing and chemo luminescence detection (S A Nesbitt et al. Anal. Biochem. 206 (1992) 267-272) (ECL, Amersham, Buckinghamshire, UK) according to the manufacturer's instructions.

### Example 7

Tetracycline Screening of Blood Donor Sera

One woman and seven men aged between 23 and 35 were selected as candidates. They had not received any anti-infectious treatment for at least one month. All were healthy and used a western standard diet. 50 ml peripheral venous blood were taken and the serum was obtained according to standard procedures. The sera were subjected to a freezing-thawing cycle before the cell culture experiments and the tetracycline determinations were carried out. Human sera were added to the cell culture media instead of FBS. Tetracycline-HCl was purchased from Fluca Chemicals (Fluca, Germany). Bakebond RP-18 solid phase extractions (SPE) columns were obtained from Mallinckrodt Baker (Phillipsburg, N.J.), solvent of HPLC quality and other chemicals were purchased from Merck (Whitehouse Station, N.J.). HPLC was carried out on a Constametric 3500 MS and RP-18 HyPURITY® ADVANCE columns from ThermoQuest (Germany). The data analysis was carried out using Chemstation software from Agilent (Germany). After preconditioning of the RP-18 columns with 2×1 ml of methanol, followed by 2×1 ml of water, 3 ml of serum containing 0.1 mole/l of citrate buffer (pH 6.8) and 0.1 mole/l of EDTA were added at a flow rate of 1 ml/min. The columns were then washed with 10 ml of water and 1 ml of methanol. Tetracycline was eluted with 4 ml of methanol containing 0.1% trifluoroacetic acid (M E Sheridan et al. J. Chromatography 434 (1988) 253-258). The eluate was dried and reconstituted in 100 μl of 0.01% oxalic acid in water/acetonitrile (98/2 v/v) adjusted with HCl at a pH of 2.0. Chromatography was carried out at room temperature and a flow rate of 0.9 mi/min. The fluorescence at 416 nm (excitation) and 515 nm (emission) was achieved by complexing the tetracycline with 0.2% (w/v) of zirconium (IV) chloride (K. De Wasch et al. Analyst 123 (1998) 2737-2741). The calibration was carried out with aqueous solutions of tetracycline HCl of 2 to 100 ng/ml with variation coefficients of 6.3% (intraday) and 8.5% (interday) for 10 ng/ml.

Results

Construction of Doxycycline-Suppressible, Autoregulated Adenoviral Vectors

Adenoviral expression plasmids containing the luciferase gene and murine scIL-12 gene subject to the control of the tetracycline-suppressible autoregulated system, pAd.3r-luc and pAd.3r-scIL-12 were generated. Following a similar procedure, plasmids were generated containing the luciferase gene and the cDNA for murine p40 and p35, which is by an internal ribosome entry site (IRES), both subject to the control of the cytomegalovirus (CMV) promoter. Recombinant E1/E3 deleted adenoviruses Ad.3r-luc, Ad.3r-scIL12, Ad.CMV-luc and Ad.CMV-p40.IRES.p35 (FIG. 2) were generated by cotransfection of the adenoviral expression plasmids with pBHG10. The plaque purification and amplification was carried out in 293 cells. Adenoviral titers were quantified by standard plaque assay techniques. The isolation, amplification and plaque assay of Ad.3r-scIL12 was up to 87 times higher in the presence of 2 μg/ml of doxycycline, indicating the toxicity of non-suppressed scIL12 expression in 293 cells (FIG. 3). In contrast, doxycycline had no influence on the titration of Ad.3r-luc.

Dose Dependent Doxycycline-Regulated Luciferase and Transactivator Gene Expression

Human colon carcinoma cells HT29 are extremely receptive for adenoviral transduction, as has been shown previously (A. Block et al. Cancer Gene Therapy 7 (2000) 438-445). These cells were infected with Ad.3r-luc at a m.o.i. (multiplicity of infection) of 30 following incubation with doxycycline in various concentrations for 24 hours. The luciferase activity was determined in cell lysates in relation to the dissolved cell protein. Even low doxycycline concentrations, such as 100 pg/ml, lead to a significant reduction in gene expression. Finally, the gene expression was maximally suppressed with doxycycline concentrations of up to 3 μg/ml (FIG. 4). This doxycycline concentration is usually used for the clinical treatment of bacterial infections. In the present experimental approach, an up to 2400 fold doxycycline-mediated suppression of the transgene expression was present.

The dose dependent, doxycycline regulated suppression of the positive feedback loop (FIG. 1) was illustrated by the detection of the tTA fusion proteins with Tet-R monoclonal (M. Gossen et al., PNAS U.S.A. 89 (1992) 5547-5551) and VP16 polyclonal antibodies (P E Pellett et al. PNAS U.S.A. 82 (1985) 5870-5874) in Western blot analysis (FIG. 5). Increasing doxycycline concentrations lead to a down regulation of the intracellular portions of tTA which correlates with a reduced luciferase gene expressions.

M.O.I.-Dependent Suppressible Luciferase Expression

HT29 cells were infected with Ad.3r-luc at a m.o.i. in a range of 0.1 to 100 following incubation in the presence or absence of doxycycline at 2 μg/ml for 24 hours. The suppression of the luciferase gene expression in lysates of Ad.3r-luc-infected HT29 cells ranged between 470 (m.o.i.: 0.3) and 2400 fold (m.o.i.: 10-100) (FIG. 6). The extent of the suppression remained constant at a high m.o.i., which is decisive for a satisfactory control of transgene expression related toxicity. Doxycycline concentrations of 2 μg/ml did not interfere with the adenoviral gene expression in HT29 cells using the constitutive CMV promoter. In order to investigate the efficiency of the Ad.3r-luc-mediated transgene expression in the absence of doxycycline, expression was compared with the expression in HT29 cells after infection with Ad.CMV-luc (FIG. 7). In HT29, Ad.3r-luc resulted in a higher gene expression than Ad.CMV-luc over all m.o.i. tested (1-100), the factor being in between 18 fold (m.o.i.: 100) and 240 fold (m.o.i.: 1).

M.O.I.-Dependent Regulated Expression of Single-Chain Murine Interleukin-12

HT29 cells were infected with Ad.3r-scIL-12 at a m.o.i. in the range of 1 to 100 and incubated in the presence or absence of 2 μg/ml of doxycycline for 24 hours. The gene expression of scIl-12 was suppressed by more than 1400 fold at a m.o.i. of 100 in the presence of doxycycline (FIG. 8). The Western blot analysis showed a transactivator (tTA) expression correlating with the IL-12 expression (FIG. 9). The non-suppressed 3r-mediated IL-12 gene expression was 11 fold (m.o.i.: 1) to 375 fold (m.o.i.: 100) higher than using the constitutive CMV promoter, assuming the same immuno reactivity of the p70 ELISA for the single chain interleukin 12 and the CMV-controlled expression of a p40/p35 heterotrimer. The bioactivity of both forms was quantified by incubation of murine splenocytes with conditioned media diluted 50 fold, following the infection of HT29 with IL-12 expressing adenoviruses (FIG. 10). A high interferon-γ (IFN-γ) secretion of splenocytes was obtained by incubation with conditioned media following the infection of HT29 with Ad.3r-sc-IL-12. This IFN-γ induction was significantly higher, compared with the infection of HT29 with Ad.CMV-p40.IRES.p35 normally used. The addition of doxycycline resulted in a suppression of IFN-γ to a background level. Also, the specific bioactivity of adenovirally expressed forms of IL-12 was analyzed in comparison with recombinant purified p40/p35 heterodimers (FIG. 11). Murine splenocytes were incubated with semi-logarithmic dilutions of recombinant heterodimeric IL-12 or IL-12 containing conditioned media, as described. The IFN-γ induction correlated with the immune activity of IL-12 in the media, as shown by p70 ELISA. The basal induction was caused by preincubation of splenocytes with anti-human CD28 antibodies leading to IL-2 expression and subsequent IFN-γ induction (C H June et al., J. Immunol. 143 (1989) 153-161). The bioactivity of the murine single-chain IL-12 fusion protein was comparable with the purified recombinant p40/p35 heterodimer. The reduced bioactivity of IL-12 which was expressed after infection with the Ad.CMV-p40.HRES.p35 usually used can be explained by inhibitory p40 homodimers (P. Ling et al. J. Immunol. 154 (1995) 116-127; S. Gillesen et al. European J. Immunol. 25 (1995) 200-206; F. Mattner et al. European J. Immunol. 23 (1993) 2202-2208).

Regulated Interleukin-12 Gene Expression In Vitro

Different cell lines of the human colon carcinoma (HT29, SkCo-1 and Colo205), pancreatic carcinoma (Aspc-1), bladder carcinoma (RT4), cervix carcinoma (HeLa), breast carcinoma (MCF-7 and BT-20) and myeloma (U266) as well as hepatocellular carcinoma (HepG2) were infected either with Ad.CMV.p40.IRES.p35 or Ad.3r.scIL-12 and incubated in the presence or absence of doxycycline. The expression of the recombinant interleukin-12 was determined, as described above, using a p70 ELISA (FIG. 12). Doxycycline-mediated suppression of the interleukin-12 expression occurred in all cell lines. In the absence of doxycycline, the 3r promoter proved to be superior to the CMV promoter in all cell lines with the exception of the U266 myeloma cell line. Interleukin-12 expression in mock-transfected cell lines was not detected. Suppression of IL-12 was 3.9 fold in U266 and ranged between 167 (HepG2) and 6000 fold (Aspc-1). With the exception of U266, where a significantly lower 3r-mediated IL-12 expression was present compared with the CMV-mediated IL-12 expression, the 3r promoter lead to a 17 fold (SkCO-1) to 4254 fold (Colo205) higher gene expression in the absence of doxycycline in all other carcinoma cell lines.

Regulated Gene Expression Following Incubation with Human Serum

In view of the low doxycycline and tetracycline (tet) concentrations required for the suppression of the transgene expression, the regulation in human colon carcinoma cells was examined in the presence of human serum in order to investigate the suitability of this approach for use in a possible clinical environment. Serum samples from healthy candidates, who had had a standard western diet, were tested for tetracycline using a standard HPLC method with a maximum sensitivity of 2 ng/ml, since tetracycline is widely used for rearing of domestic cattle and a contamination of foods can be assumed. HPLC did not show any significant tetracycline concentrations in any of the samples tested. HT29 colon carcinoma cells which were infected after incubation with these human sera with Ad.3r-luc (m.o.i.: 30) did not show any significant differences regarding the transgene expression compared with certified, tetracycline-free fetal bovine serum (FIG. 13). This observation reflects tetracycline concentrations in human serum samples of less than 10 pg/ml. As expected, the supplementation of these human sera with doxycycline (2 μg/ml) led to an extremely efficient suppression of transgene expression.

### DESCRIPTION OF THE FIGURES

FIG. 1. Principle of an autoregulated tetracycline-dependent transactivator expression. The bi-directional tet-responsive promoter controls both the transgene and the transactivator expression. Binding of the transactivator in the absence of tetracycline or doxycycline results in an amplification of the transactivator expression by a positive feedback loop as well as in an induction of the transgene expression. tTA, tet repressor and VP16 fusion proteins; TKmin, minimal thymidine-kinase promoter; CMVmin, minimal cytomegalovirus promoter; TetO₇, heptamerized Tet operator.

FIG. 2. Adenoviral vector maps. The autoregulated tetracycline expression cassette is inserted into the ΔE1 region of the adenoviral genome. To avoid cryptic splicing and to obtain RNA stability (Ad.3r-luc and Ad.3r-scIL12), an intron was inserted upwards from the activator and the luciferase or interleukin-12 gene from the mouse. In addition, recombinant, adenoviral vectors for the expression of the luciferase or the heterodimeric interleukin-12 gene of the mouse was constructed subject to the control of the CMV promoter (Ad.CMV-luc and Ad.CMV-p40.IRES.p35). E1 and E3, early regions of the adenoviral genomes; IRES, internal ribosome entry sites; CMV, cytomegalovirus promoter; TK, thymidine kinase promoter.

FIG. 3. Plaque assay of Ad.3r-scIL12 in the presence and absence of doxycycline at a concentration of 2 μg/ml. The titration of Ad.3r.scIL12 in 293 cells results in a considerably higher yield if the expression of the transgene is suppressed by the addition of doxycycline. Dox, doxycycline.

FIG. 4. Dose-dependent luciferase expression following infection of HT29 colon carcinoma cells with Ad.3r-luc followed by different concentrations of the tetracycline derivative doxycycline.

FIG. 5. Western blot analyses of transactivator show the positive feedback loop after adenoviral infection of HT29 cells and incubation with different quantities of doxycycline. The figure shows the suppression of the expression of the tTA fusion protein in the presence of doxycycline. dox, doxycycline.

FIG. 6. Suppression of the luciferase gene expression following infection of HT29 cells with different multiplicities of infection (m.o.i.). Doxycycline-regulated gene expression is achieved in a large range of infection from at least 0.1 to 100 m.o.i. which results in a 470 to 2400 fold suppression of the luciferase expression.

FIG. 7. Comparison of the 3r-mediated transgene expression with the constitutive cytomegalovirus promoter. HT29 cells were infected with Ad.3r-luc or Ad. CMV-luc at different m.o.i., followed by incubation in doxycycline-free medium.

FIG. 8. Interleukin-12 expression in HT29 cells following infection with Ad.3r-scIL-12 in the presence or absence of doxycycline (2 μg/ml) or Ad.CMV.mIL12 at different m.o.i. As shown with luciferase-expressing adenoviral vectors, a considerably higher interleukin-12 expression is present in HT29 when the 3r promoter is used. The addition of doxycycline leads to a suppression of the transgene expression below the level achieved with Ad.CMV-p40.IRES.p35 at the same m.o.i.

FIG. 9. Western blot analysis of tTA transactivator gene expression in the presence or absence of doxycycline following infection with Ad.3r-scIL12 at different m.o.i. Both domains of the tTA fusion protein were detected with the TetT and VP16 antibodies. The expression of the tTA fusion protein correlates with the m.o.i. used. Addition of doxycycline at a concentration of 2 μg/ml results in a suppression of the tTA expression. TetR, tetracycline repressor; VP16, Herpes simplex virus transcriptional activation domain.

FIG. 10. Induction of the interferon-γ expression following incubation of splenocytes with conditioned supernatant of infected HT29 cells. 10⁶ HT29 cells were infected with Ad.3r-scIL12 (+/−dox) or Ad.CMV-p40.IRES.p35 at an m.o.i. of 30 for 24 h. Infection of HT29 with Ad.3r-scIL12 resulted in a strong interferon-γ induction in comparison with a Ad.CMV-p40.IRES.p35 infection. Addition of doxycycline resulted in a decrease in interferon-γ to a background level in this test.

FIG. 11. Comparison of the interferon-γ induction by adenovirally expressed single chain and/or heterodimeric interleukin-12 and purified recombinant interleukin-12. Interleukin-12 in the conditioned supernatant of infected HT29 cells was determined by p70-mIL12 ELISA. Mouse splenocytes were then incubated with serial dilutions of either adenovirally expressed or recombinant interleukin and the induced interferon-γ was quantified using a mIFN-γ ELISA. The bioactivity immune reactivity of single chain interleukin-12 was comparable with recombinant purified heterodimeric interleukin-12. The specific bioactivity of adenovirally produced heterodimeric interleukin-12 (Ad.CMV-p40.IRES.p35) seems to be lower, probably as a result of inhibitory p40 homodimers.

FIG. 12. Interleukin-12 expression in different cell lines following infection with either Ad.CMV-p40.IRES.p35 or Ad.3r-scIL12 in the presence or absence of doxycycline. Different levels of transgene expression are partly due to differences in transduction efficiency. With the exception of the U266 myeloma cell line, the 3r-mediated gene expression was substantially higher than CMV-mediated expression.

FIG. 13. Incubation of Ad.3r-luc infected HT29 colon carcinoma cells with human sera instead of certified tetracycline-free fetal bovine serum. No significant differences arose when using human serum of volunteers with a standardized western diet in comparison with certified tetracycline-free fetal calf serum. These data suggest a tetracycline-concentration in human volunteers of less than 50 pg/ml. The supplementation of human sera with doxycycline (2 μg/ml) resulted in a suppression of the transgene expression, as shown above. FCS, fetal calf serum.

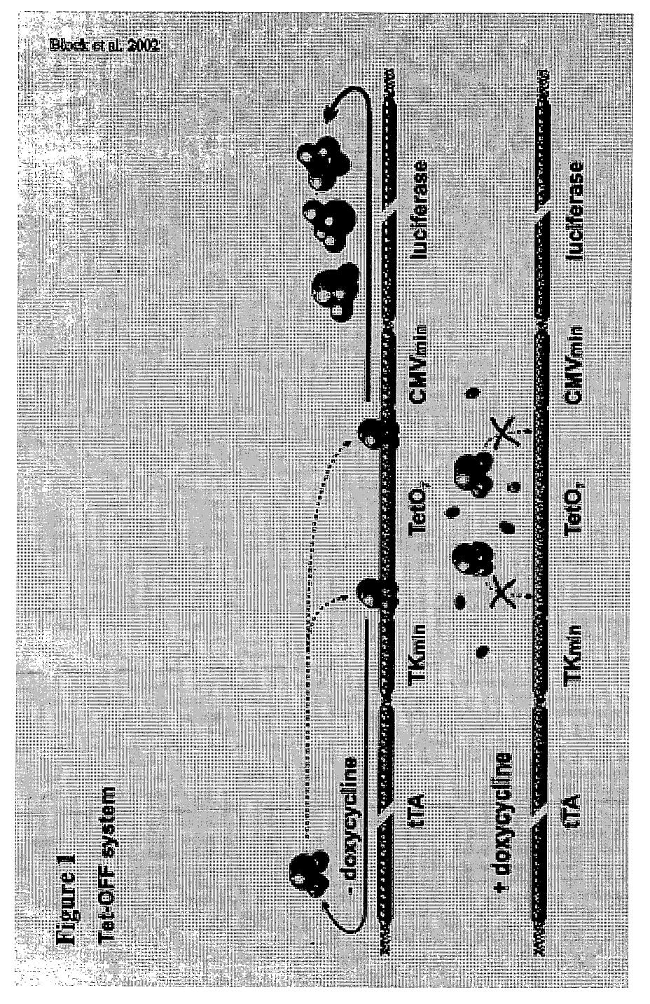

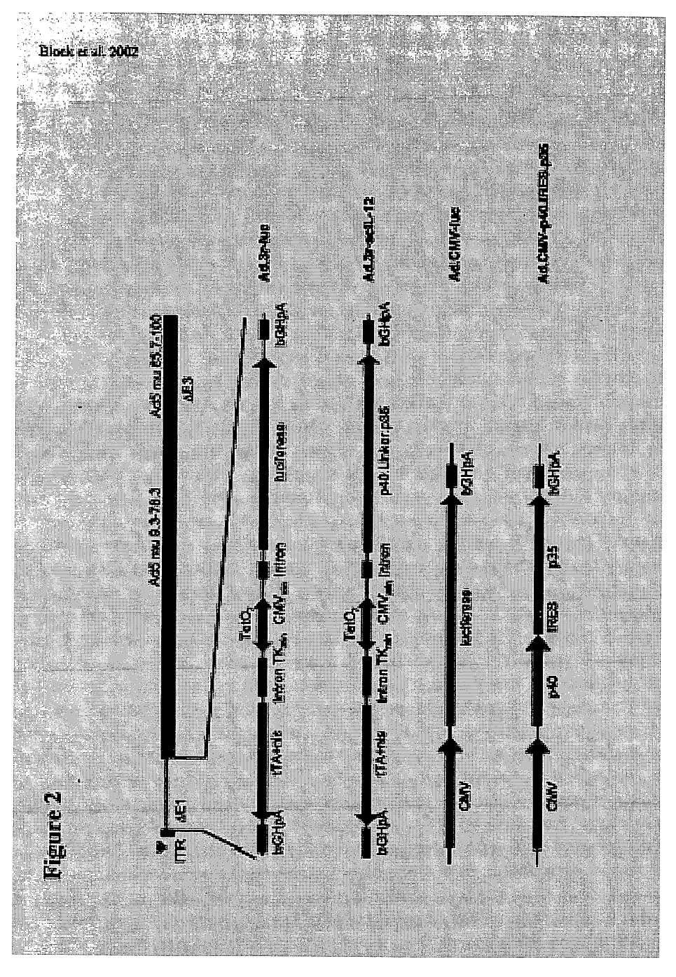

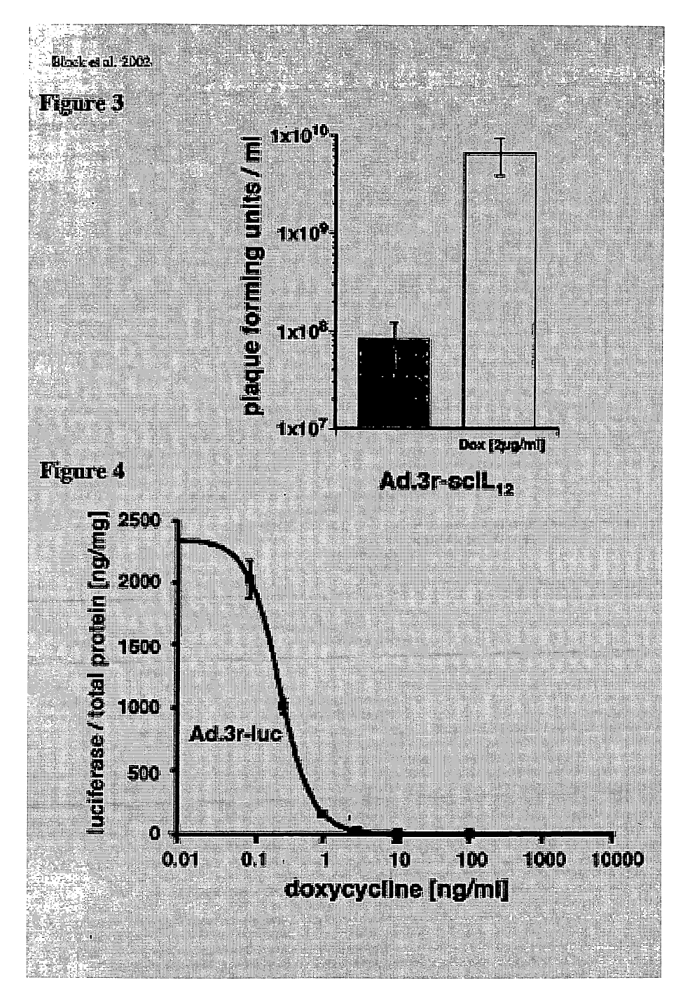

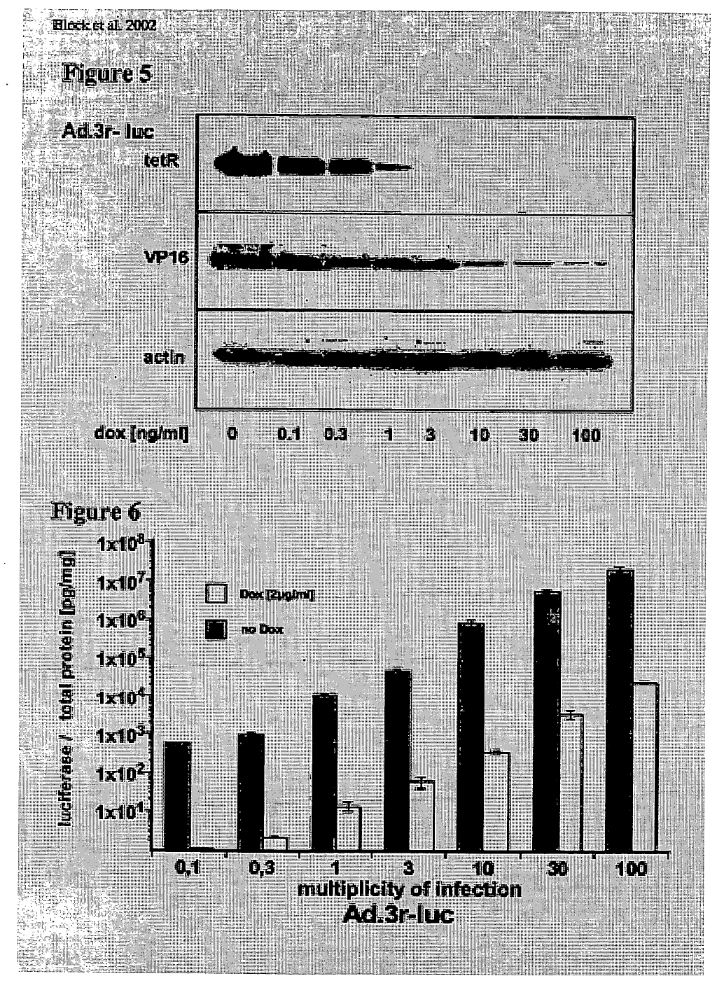

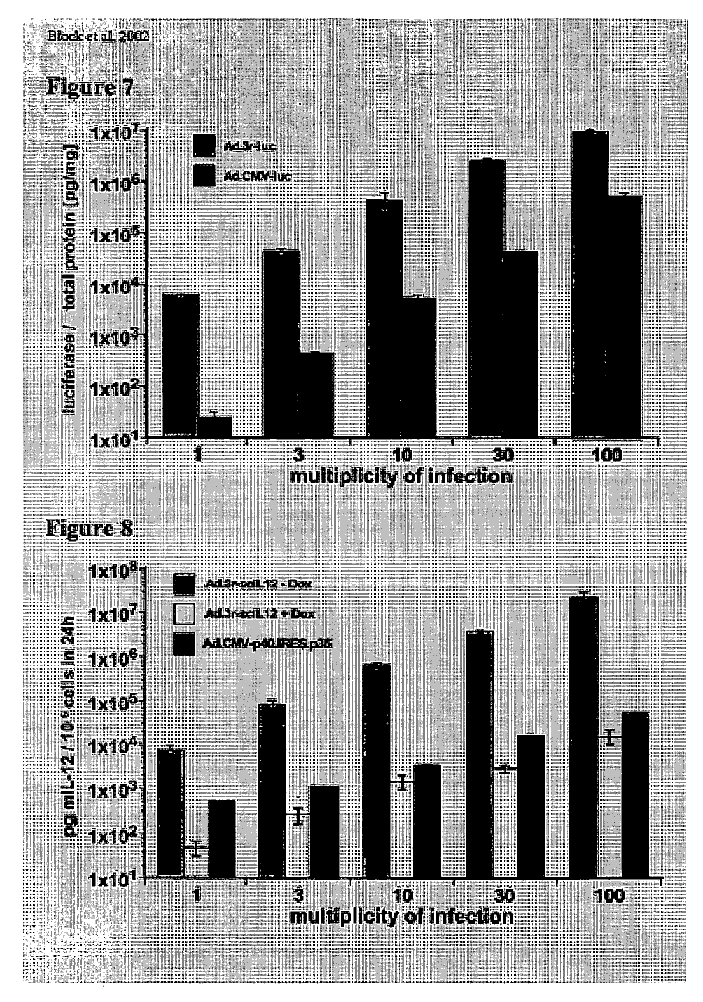

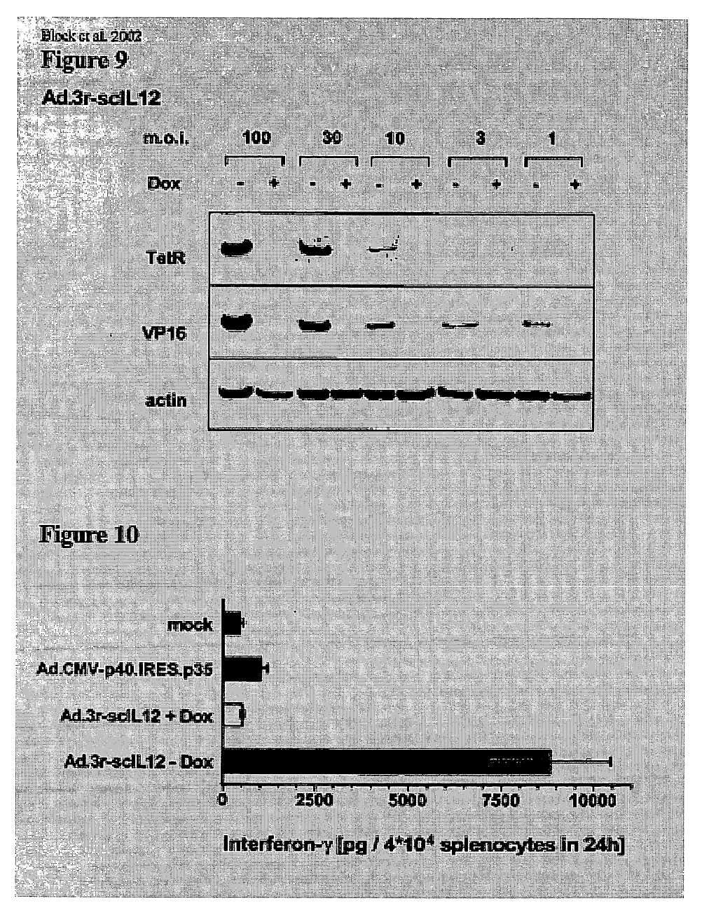

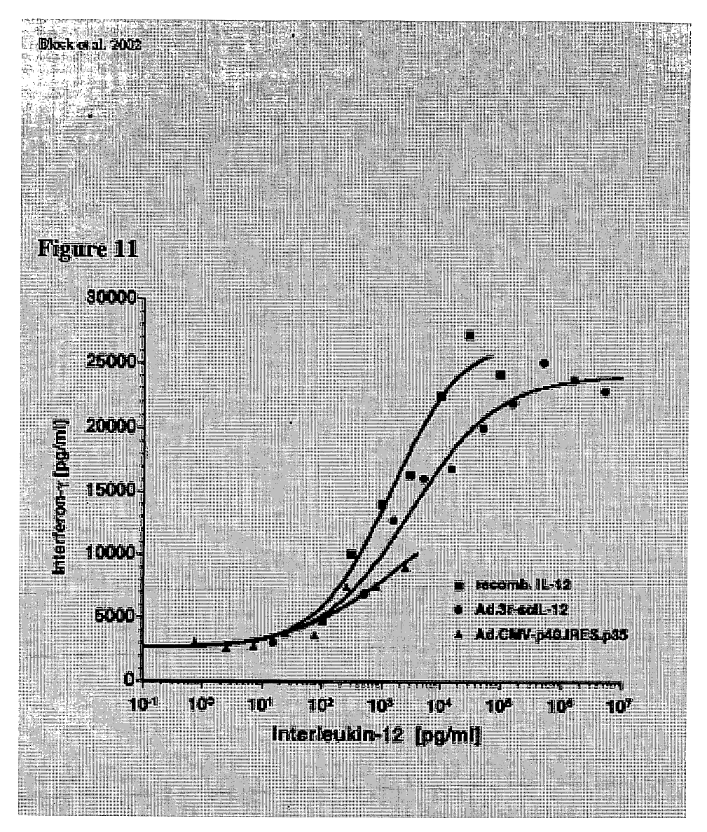

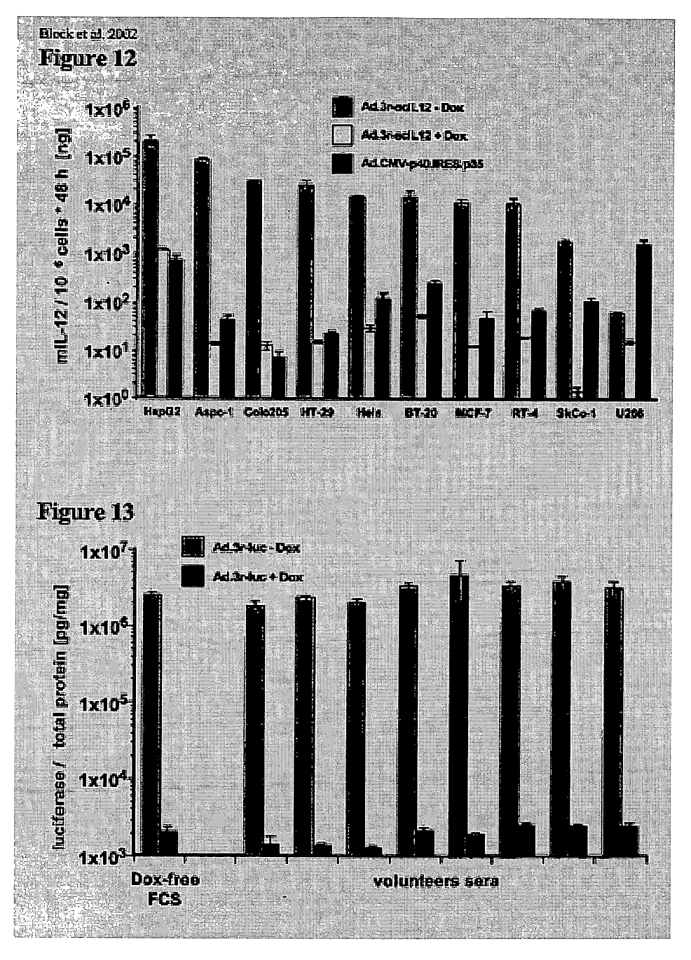

1. Recombinant viral vector which contains an insert exhibiting the general structure
tTA-intron¹-TK⁺-TetO₇-CMV⁺-intron²-transgene
in which
TetO₇ is the heptamerized tetracycline operator
TK⁺ is the minimal thymidine kinase promoter
tTA is a nucleic acid sequence which encodes a fusion protein from the repressor protein inducible by tetracycline and the transcriptional activation domain of the Herpes simplex virus VP16,
CMV⁺ is the minimal cytomegalovirus promoter and
Transgene is a nucleic acid sequence which codes for a non-viral protein
Intron¹ is any desired non-encoding nucleic acid sequence with a length of O to approximately 1000 bp and
Intron² is any desired non-encoding nucleic acid sequence with a length of O to approximately 1000 bp.

2. Vector according to claim 1 characterized in that the insert is inserted into the viral vector genome in reverse orientation.

3. Vector according to claim 1 or 2 characterized in that the positions of tTA and transgene are inverted in the insert.

4. Vector according to claims 1 to 3 characterized in that the insert contains an additional lac repressor (lacR) between “CMV⁺” and “intron²” or between “intron²” and “transgene”.

5. Vector according to claims 1 to 4 characterized in that the transgene is a nucleic acid sequence encoding a fluorescence protein, luciferase, interleukin-12 (IL-12), interleukin-18 (IL-18), interleukin-2 (IL-2), tumor necrosis factor α (TNF-α) or interferon-γ (IFN-γ).

6. Vector according to claim 5 characterized in that IL-12 is a single chain interleukin-12.

7. Vector according to claims 1 to 6 characterized in that the virus is an adenovirus, an adeno-associated adenovirus (AAV), a retrovirus, in particular a human immunodeficiency virus (HIV), a Herpes simplex virus, a Hepatitis B virus or Hepatitis C virus.

8. Vector according to claims 1 to 7 characterized in that the insert is cloned into the E1 and/or the E3 region of a recombinant adenovirus.

9. Vector according to claims 1 to 8 characterized in that it is obtainable by homologous recombination of a viral plasmid and an expression plasmid with the nucleic acid sequence represented in SEQ ID NO:1, SEQ ID NO:2 or SEQ ID NO:3.

10. Expression plasmid with the nucleic acid sequence represented in SEQ ID NO:4 or SEQ ID NO:5.

11. Use of a plasmid according to claim 10 for the production of a vector according to claims 1 to 9.

12. Use of the vector according to claims 1 to 9 for the in vitro gene expression in eukaryotic cell lines.

13. Use of the vector according to claim 1 to 9 in the case of which “transgene” encodes a therapeutically effective protein, for the preparation of a medicament for gene therapy.

14. Use according to claim 13 in which the transgene is IL-2, IL-12, IL-18, TNF-α or INF-γ, and the gene therapy is the gene therapy of malignant diseases.

15. Use according to claim 14 characterized in that the malignant disease is a solid tumor.

16. Use according to claims 12 to 15 characterized in that the gene expression is regulated with doxycycline, tetracycline, oxytetracycline, chlorotetracycline, demeclocycline, methacycline or minocycline.

17. Use of the vectors according to claims 1 to 9 in which “transgene” encodes a reporter protein, for the detection of tetracycline or a derivative thereof in biological, food chemical or similar samples.

18. Use according to claim 17 characterized in that the derivative is doxycycline.
